(19) United States
(12) Patent Application Publication
(10) Pub. No.: US 2022/0151959 A1
(43) Pub. Date: May 19, 2022

(54) METHOD FOR TREATING ALLERGIC SKIN DISEASE OR PRURITUS CUTANEOUS

(71) Applicant: KOREA RESEARCH INSTITUTE OF BIOSCIENCE AND BIOTECHNOLOGY, Daejeon (KR)

(72) Inventors: Bo Yeon KIM, Daejeon (KR); Joonsung HWANG, Daejeon (KR); In Ja RYOO, Daejeon (KR); Nak Kyun SOUNG, Daejeon (KR); Jong Seog AHN, Daejeon (KR); Min Sik WOO, Daejeon (KR); Kyung Ho LEE, Daejeon (KR); Hee Gu LEE, Daejeon (KR); Hyunjoo CHA, Daejeon (KR)

(73) Assignee: KOREA RESEARCH INSTITUTE OF BIOSCIENCE AND BIOTECHNOLOGY, Daejeon (KR)

(21) Appl. No.: 17/433,829

(22) PCT Filed: Feb. 24, 2020
(86) PCT No.: PCT/KR2020/002596

§ 371 Date: Aug. 25, 2021

(30) Foreign Application Priority Data
Feb. 26, 2019 (KR) 10-2019-0022315

(51) Int. Cl. A61K 31/165; A61K 8/42; A61Q 19/00; A61P 37/08; A61P 17/00

(52) U.S. Cl. CPC A61K 31/165; A61K 8/42; A61P 17/00; A61P 37/08; A61Q 19/007

(57) ABSTRACT

A composition containing colchicine and its uses are disclosed. The composition enhances the expression amount of filaggrin that is highly related to the onset of allergic skin diseases, enhances skin moisturizing effects to thereby improve the skin barrier function, and in addition, controls inflammatory responses by allergic skin diseases. Therefore, methods for prevention and/or treatment of allergic skin diseases and/or pruritus cutaneous and skin moisturization and skin condition improvement are disclosed.

### TECHNICAL FIELD

The present invention relates to a composition for treating an allergic skin disease and/or pruritus cutaneous, including colchicine.

### BACKGROUND ART

Skin is anatomically positioned in the outermost of a body, and blocks the direct entry of pathogenic microorganisms, viruses, chemicals, and the like in the air to protect the body from the external environment and performs an important barrier function to prevent excessive leakage of body moisture. A skin tissue forms a layer structure of a basal layer, a spinous and granular layer, and a cornified layer, and a particular expression marker in each layer structure is well known. Among them, keratin (K1) and keratin 10 (K10) are expressed in a spinous and granular layer and filaggrin is expressed in the top/epithelium including a cornified layer, and these are one of the main proteins which are essential for forming a barrier function specific to the skin described above.

Among the skin-related diseases, a disease which is the most problematic and lacks an adequate therapy up to date may be atopic dermatitis. According to the data released by the Korean National Health Insurance Corporation, the number of patients treated for atopic dermatitis is more than 1 million, and in particular, an incidence rate in infancy is high such that the number of infant patients aged 0 to 4 years is close to 320,000 which is more than ⅓ of all patients and the number of child patients under 9 years of age is half of the total number of patients. In nine countries including the US, the UK, and China around the world, the number of atopic dermatitis patients is expected to increase to 140 million in 2022, and the related therapeutics market scale is also expected to grow to about KRW 8 trillion. As such, atopic dermatitis shows an increasing trend in a prevalence rate worldwide as well as domestically, and the reason for classifying the atopic dermatitis as a modern incurable disease may be the absence of an accurate diagnosis for accurately diagnosing the etiology and a correspondingly customized therapy.

For example, a steroid-type atopic dermatitis inhibitor is currently used as a therapeutic agent, but due to the absence of a diagnosis for accurately diagnosing the etiology and a correspondingly customized accurate therapy, not a fundamental solution of atopic dermatitis but only a temporary palliative effect is shown and also there is a side effect of tolerance, and thus, the use thereof is much limited.

Recently, a correlation between atopic dermatitis and a filaggrin gene abnormality was revealed, and in particular, in Europe, a filaggrin gene mutation was detected in more than half of the total number of atopic patients. Also, in Japan, about 25% or more of atopic patients had a filaggrin gene mutation, and in various Asian countries including China and Korea, a filaggrin gene mutation was detected in patients with dermatitis including atopic dermatitis. It has been reported that the filaggrin gene abnormality causes decreased filaggrin protein expressed in skin and a loss of a skin barrier function, and promotes penetration of an antigen such as an allergic stimulator to cause dermatitis. In reality, among atopic patients, patients having filaggrin abnormality easily progress to allergic diseases such as asthma and rhinitis, and diagnostics to determine whether there is a filaggrin gene abnormality and customized therapy establishment are desperately needed.

Meanwhile, colchicine is an alkaloid compound derived from plants of the lily family including Colchicum autumnale. The medical usefulness thereof has been recognized for centuries, and its use is expanded from an oral drug used for treating acute gouty arthritis to a therapeutic agent of Familial Mediterranean Fever (FMF).

### DISCLOSURE

### Technical Problem

The present researchers confirmed that colchicine adjusts an expression amount of filaggrin in skin, thereby enhancing a barrier function specific to the skin, enhancing a water retention, and also showing an effect of preventing, treating, and improving a disease in an atopic dermatitis model, and thus, completed the present invention.

Thus, the present invention is to provide the following:

An object of the present invention is to provide a pharmaceutical composition for preventing or treating an allergic skin disease and/or pruritus cutaneous, including colchicine or a pharmaceutically acceptable salt thereof.

Another object of the present invention is to provide a cosmetic composition for skin moisturization, including colchicine or a pharmaceutically acceptable salt thereof.

Another object of the present invention is to provide a quasi-drug composition for skin moisturization, including colchicine or a pharmaceutically acceptable salt thereof.

Still another object of the present invention is to provide a skin external preparation composition for skin moisturization, including colchicine or a pharmaceutically acceptable salt thereof.

### Technical Solution

In one general aspect, a pharmaceutical composition for preventing or treating an allergic skin disease and/or pruritus cutaneous includes: colchicine or a pharmaceutically acceptable salt thereof.

The present invention provides a pharmaceutical composition for preventing or treating an allergic skin disease, pruritus cutaneous, or both of them, including colchicine or a pharmaceutically acceptable salt thereof.

The composition according to the present invention shows an excellent effect in prevention and treatment of an allergic skin disease, pruritus cutaneous, or both of them, by operation effects, such as enhancing an expression amount of filaggrin which is highly related to the onset of an allergic skin disease, enhancing a skin moisturizing effect to improve a skin barrier function, and also adjusting an inflammatory response of the allergic skin disease and decreasing skin itching.

The composition of the present invention includes colchicine as an effective component.

Colchicine is a compound having a structure of the following Chemical Formula 1:

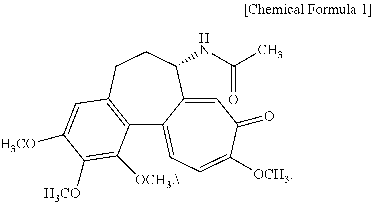

In the present invention, a pharmaceutically acceptable salt refers to a salt which is commonly used in the pharmaceutical industry, and, an example thereof includes an inorganic ion salt prepared with calcium, potassium, sodium, magnesium, and the like, an inorganic acid salt prepared with hydrochloric acid, nitric acid, phosphoric acid, bromic acid, iodic acid, perchloric acid, sulfuric acid, and the like, an organic acid salt prepared with acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like, a sulfonic acid salt prepared with methane sulfonic acid, ethane sulfonic acid, benzene sulfonic acid, p-toluene sulfonic acid, naphthalene sulfonic acid, and the like, an amino acid salt prepared with glycine, arginine, lysine, and the like, amine salt prepared with trimethylamine, triethylamine, ammonia, pyridine, picoline, and the like, and the like, but the kind of the salt of the meaning in the present invention is not limited to the salts listed.

The composition including colchicine as an effective component has an effect of preventing and treating an allergic skin disease and/or pruritus cutaneous and also may show a skin moisturizing effect.

The term “allergic skin disease” in the present invention refers to a pathological symptom caused by an allergic reaction mediated by mast cell activation such as mast cell degranulation, and the allergic skin disease representatively includes atopic dermatitis, allergic dermatitis, contact dermatitis, and the like. The atopic dermatitis shows a symptom such as dry eczema skin or papule, and epidermal hyperplasia, cuticularization, and accumulation of lymphocytes and mast cells, and the like are confirmed in a lesion sample of an atopic patient. An atopic dermatitis patient may suffer from severe pruritus cutaneous, which causes inflammation of skin lesions to further worsen clinical symptoms.

The term “pruritus cutaneous” in the present invention is a disease including pruritus caused by decreased antibacterial activity and deteriorated barrier function due to a decrease of a lipid content in the cornified layer of skin or pruritus caused by an external stimulus such as a temperature change, chemicals, and an electrical stimulation.

The term “anti-allergic” in the present invention refers to inclusion of improvement (alleviation of symptoms), treatment, and prevention (inhibition or delay of pathogenesis) of an allergic skin disease.

In an exemplary embodiment of the present invention, colchicine enhances expression of filaggrin in skin and enhances a skin moisturizing effect to show an excellent improvement effect in terms of a skin barrier index and a skin composite index. In addition, colchicine greatly inhibits an inflammatory response in an allergic skin disease model, inhibits epidermal hyperplasia, cuticularization, and the like, adjusts inflammatory cytokine, and decreases skin itching to show an excellent prevention, treatment, and improvement effect for an allergic skin disease and/or pruritus cutaneous.

The term “inflammatory cytokine” in the present invention refers to cytokine causing an inflammatory response occurring in the body, and is used in an ordinary sense in the art to which the present invention pertains. For example, IL-2, IL-4, IL-13, and the like may act as an inflammatory cytokine to cause an allergic skin disease.

The pharmaceutical composition of the present invention may further include one or more effective components showing an anti-inflammatory activity.

The composition of the present invention may further include a pharmaceutically acceptable additive, and as the pharmaceutically acceptable additive, starch, gelatinized starch, microcrystalline cellulose, milk sugar, povidone, colloidal silicon dioxide, calcium hydrogen phosphate, lactose, mannitol, maltose, Gum arabic, pregelatinized starch, corn starch, powdered cellulose, hydroxypropyl cellulose, Opadry, sodium starch glycolate, carnauba lead, synthetic aluminum silicate, stearic acid, magnesium stearate, aluminum stearate, calcium stearate, sucrose, dextrose, sorbitol, talc, and the like may be used. It is preferred that pharmaceutically acceptable additive according to the present invention is included at 0.1 to 90 parts by weight with respect to the composition, but is not limited thereto.

The composition of the present invention may be administered as various oral and parenteral formulations in real clinical administration, and when being formulated, may be prepared using a diluent or an excipient such as a filler, an extender, a binder, a wetting agent, a disintegrant, and a surfactant. Preferably, in the present invention, the composition may be used as a parenteral agent, in particular, a coating agent.

The composition of the present invention may be administered orally or parenterally depending on the method to be desired, and when parenterally administered, transdermal administration, external application to skin, application to skin, intraperitoneal injection, intrarectal injection, subcutaneous injection, intravenous injection, intramuscular injection, or intrathoracic injection may be selected, and for example, the composition may be transdermally administered. A dosage range is varied depending on weight, age, gender, health condition, diet, administration time, administration method, excretion rate, disease severity, and the like of a patient. Preferably, the composition according to the present invention may be administered to a human body by parenteral administration, skin external use, or application to skin.

When the composition is prepared as a transdermal administration agent, it may be prepared in the form of an ointment, a cream, a lotion, a gel, an external solution, a paste agent, a liniment, an aerosol agent, and the like.

For preparation of the pharmaceutical composition is known in the art, and specifically, documents such as Remington's Pharmaceutical Sciences (19th ed., 1995) may be referred. The above document is regarded as a part of the present specification.

The composition according to the present invention is administered in a pharmaceutically effective amount. In the present invention, “pharmaceutically effective amount” refers to an amount sufficient to treat a disease at a reasonable benefit/risk ratio applicable to medical treatment, and an effective dose level may be determined by factors including the kind and severity of the disease of a patient, an activity of a drug, sensitivity to a drug, administration time, administration route and releasing rate, treatment period, and a simultaneously used drug, and other factors well known in the medical field. The composition of the present invention may be administered as an individual therapeutic agent, or in combination with other therapeutic agents, sequentially or simultaneously with the traditional therapeutic agent, and as a single dose or multiple doses. It is important to administer the composition in an amount to obtain a maximum effect with a minimum amount without any side effect, considering all of the above elements, and this will be easily determined by those skilled in the art.

Specifically, the effective amount of the compound according to the present invention may be varied with the age, the gender, and the weight of a patient, and generally, may be administered at 0.1 mg to 100 mg, preferably 0.5 mg to 10 mg per 1 kg of body weight daily, every other day, or 1 to 5 times a week, or may be divided into 1 to 5 doses per day and administered. However, since the amount may be increased or decreased depending on administration route, severity, gender, weight, age, and the like, the administration amount in no way limits the scope of the present invention.

In another general aspect, a cosmetic composition for skin moisturization includes colchicine or a pharmaceutically acceptable salt thereof.

The term “skin moisturization” in the present invention refers to increasing moisture in skin and maintaining a moist condition.

The cosmetic composition for skin moisturization according to the present invention has an excellent skin moisturizing effect of suppressing or decreasing skin moisture loss, and adjusts expression of factors such as filaggrin, involucrin, and loricrin which are moisturizing-related factors to show an excellent effect as a skin moisturizing composition.

The cosmetic composition according to the present invention may be prepared as one or more formulations selected from the group consisting of a solution, an external ointment, a cream, a foam, a nourishing lotion, a softening lotion, a pack, a softener, a milky lotion, a makeup base, an essence, a soap, a liquid detergent, a bath preparation, a sunscreen cream, a sun oil, a suspension, an emulsion, a paste, a gel, a lotion, a powder, a surfactant-containing cleansing, an oil, a powder foundation, an emulsion foundations, a wax foundations, a patch, and a spray, but is not limited thereto.

In addition, the cosmetic composition of the present invention may further include one or more cosmetically acceptable carriers combined with common skin cosmetics, and as a usual component, for example, oil, water, surfactant, humectant, lower alcohol, thickener, chelating agent, colorant, preservative, fragrance, and the like may be properly combined, but is not limited thereto.

A cosmetically acceptable carrier included in the cosmetic composition of the present invention is various depending on the formulation.

When the formulation of the present invention is an ointment, a paste, a cream, or a gel, animal oil, vegetable oil, wax, paraffin, starch, tragacanth, cellulose derivatives, polyethylene glycol, silicone, bentonite, silica, talc, zinc oxide, or a mixture thereof as a carrier component may be used.

When the formulation of the present invention is a powder or spray, lactose, talc, silica, aluminum hydroxide, calcium silicate, polyamide powder, or a mixture thereof may be used as a carrier component, and in particular, when the formulation is a spray, a propellant such as chlorofluorohydrocarbon, propane/butane, or dimethyl ether may be further included.

When the formulation of the present invention is a solution or an emulsion, a solvent, a solubilizer, or an emulsifier is used, for example, water, ethanol, isopropanol, ethyl carbonate, ethyl acetate, benzyl alcohol, benzyl benzoate, propylene glycol, and 1,3-butylglycol oil may be used, and in particular, cottonseed oil, peanut oil, corn germ oil, olive oil, castor oil, and sesame oil, glycerol aliphatic ester, polyethylene glycol, or fatty acid ester of sorbitan may be used, as a carrier component.

When the formulation of the present invention is suspension, a liquid diluent such as water, ethanol, or propylene glycol, a suspension such as ethoxylated isostearyl alcohol, polyoxyethylene sorbitol ester, and polyoxyethylene sorbitan ester, microcrystalline cellulose, aluminum methahydroxide, bentonite, agar, or tragacanth, and the like may be used as a carrier component.

When the formulation is a soap, an alkali metal salt of a fatty acid, a fatty acid hemiester salt, a fatty acid protein hydrolysate, isethionate, lanoline derivatives, aliphatic alcohol, vegetable oil, glycerol, sugar, and the like may be used as a carrier component.

When the formulation of the present invention is a surfactant-containing cleansing, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanoline derivatives, or ethoxylated glycerol fatty acid ester, and the like may be used as a carrier component.

The cosmetic composition according to the present invention may include 0.01 to 20 wt % of colchicine with respect to the total weight of the composition.

In another general aspect, a quasi-drug composition for skin moisturization includes colchicine.

In the present invention, the quasi-drug composition is a quasi-drug composition for the purpose of skin moisturization.

The term “quasi-drug” used in the present invention refers to an article being less effective than a drug, among articles used for diagnosing, treating, improving, alleviating, curing, or preventing a human or animal's disease, and for example, according to the pharmaceutical affairs act, a quasi-drug includes fiber/rubber products used for treatment or prevention of human or animal diseases, a non-appliance, a non-machinery, or similar articles which have insignificant influences on or do not directly act upon human bodies, a medication for sterilization, insecticide, and the like for preventing infectious diseases, excluding articles used for a drug.

The kind or formulation of quasi-drug composition of the present invention is not particularly limited, but preferably, may be a disinfectant cleanser, a shower foam, a mouthwash (mouth freshener), a wet wipe, a detergent soap, a hand wash, a humidifier filler, a mask, an ointment, a filter filler, or the like.

When the composition of the present invention is included in the quasi-drug for skin moisturization, the composition may be included as it is or used with other quasi-drug components, and may be properly used according to a usual method. A mixing amount of the effective component may be appropriately determined depending on a use purpose, and the quasi-drug according to the present invention may contain 0.01 to 20 wt % of colchicine with respect to the total weight of the composition.

In another general aspect, a skin external preparation composition for skin moisturization includes colchicine.

In the present invention, the skin external preparation composition is a skin external preparation composition for the purpose of skin moisturization.

An example of the skin external composition preparation according to the present invention may include an ointment, a lotion, a solubilized phase, a suspension, an emulsion, a cream, a gel, a spray, a gel patch, a plaster preparation, a patch, a liquid painkiller, or the like, but is not limited thereto, and may be combined with any base material known in the art. The skin external preparation composition according to the present invention may include 0.01 to 20 wt % of colchicine with respect to the total weight of the composition.

In another general aspect, a method of treating an allergic skin disease and/or pruritus cutaneous includes applying a therapeutically effective amount of colchicine to the skin of a subject.

In another general aspect, a method of moisturizing skin includes applying an effective amount of colchicine to the skin of a subject.

In another general aspect, a method of improving skin includes applying an effective amount of colchicine to the skin of a subject.

The subject refers to an animal, and typically a mammal which may show a beneficial effect with the treatment using colchicine of the present invention. A preferred example of the subject may include primates such as a human. In addition, the subject may include all of subjects having symptoms of the allergic skin disease or being at risk of having the symptoms as such, subjects in need of skin moisturization such as asteatosis and/or pruritus cutaneous, and the like.

In another general aspect, a use of colchicine in production of a pharmaceutical for treating an allergic skin disease and/or pruritus cutaneous is provided.

In another general aspect, a composition includes colchicine for use for treating an allergic skin disease and/or pruritus cutaneous.

In still another general aspect, a use of colchicine for treating an allergic skin disease and/or pruritus cutaneous is provided.

### Advantageous Effects

The composition according to the present invention enhances an expression amount of filaggrin which is highly related to the onset of an allergic skin disease, enhances a skin moisturizing effect to improve a skin barrier function, and also adjusts an inflammatory response of the allergic skin disease and the like to decrease skin itching, thereby showing an excellent effect in prevention and treatment of an allergic skin disease and/or pruritus cutaneous. Accordingly, the composition may be variously used as a pharmaceutical composition for preventing or treating an allergic skin disease and/or pruritus cutaneous; cosmetics for skin moisturization, a skin external preparation, and a quasi-drug composition.

### DESCRIPTION OF DRAWINGS

FIG. 1 shows results of confirming an increase in an expression amount of filaggrin by the colchicine treatment of the present invention.

FIG. 2 shows results of a decrease in a moisture loss by the colchicine treatment of the present invention; and an increase in a skin barrier index and a skin composite index.

FIG. 3 shows results of confirming inflammation inhibition by the colchicine treatment of the present invention, in an atopic dermatitis model.

FIG. 4 shows results of confirming inflammation inhibition by the colchicine treatment of the present invention by Hematoxylin and Eosin staining, in an atopic dermatitis model.

FIG. 5 shows results of confirming a decrease in expression of IL-4 and IL-13 by the colchicine treatment of the present invention.

### BEST MODE

Hereinafter, the present invention will be described in detail by Examples. However, the following Examples are only illustrative of the present invention, and do not limit the present invention in any way.

### Example 1. Confirmation of Skin Improvement Effect of Colchicine

(1) Confirmation of Change in Expression Amount of Filaggrin Depending on Colchicine Treatment

Filaggrin, which is a kind of epidermal protein, is a protein which performs a function of allowing skin to maintain moisture and moisturizing components and also is essential for inhibiting penetration of antigens, bacteria, or toxins from outside. First, profilaggrin which is a precursor of filaggrin is secreted and then is decomposed to a filaggrin monomer, and agglomerates keratin intermediate microfibers in a cornified layer to form an insoluble keratin matrix. Based on the matrix, a cornified cell envelope is formed just below a cell membrane of keratinocytes. Then, filaggrin is further decomposed to NMF which is a hydrophilic amino acid. NMF is a strong moisturizer and plays an important role in moisture supply to the cornified layer and lowers the pH of the cornified layer to have an antibacterial effect. In addition, it has been reported that a decreased expression of filaggrin causes a loss of skin barrier function and promotes antigen penetration in an allergic skin disease such as atopic dermatitis, thereby worsening the allergic skin disease.

Thus, in the present invention, an expression amount of filaggrin by applying colchicine was confirmed, thereby confirming a skin improvement effect.

Specifically, colchicine was continuously applied on back and ear skin areas of laboratory mice at a low concentration of 0.04% (w/w) and at a high concentration of 0.1% (w/w) twice a week for 4 weeks or more. After completing the application experiment, a skin tissue was collected from each mouse and crushed with a tissue crusher to extract RNA. The crushed tissue powder was dissolved in a Trizol reagent solution (Sigma) and RNA was extracted using a RNeasy mini kit (Qiagen). The extracted RNA was reverse-transcribed to cDNA using IMPROM-II™ Reverse Transcription kit (Promega), and then an amount of filaggrin present in the skin tissue was measured by a quantitative PCR (qPCR) method using CFX96™ Real-Time PCR Detection System (Bio-Rad).

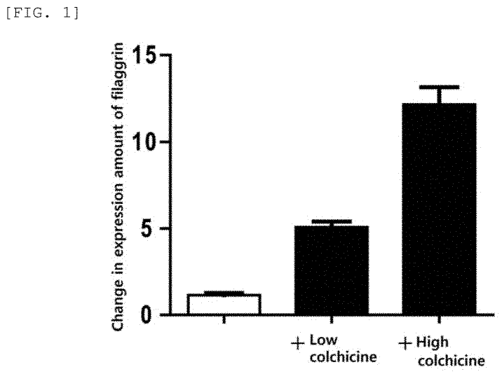

The results are shown in FIG. 1.

As confirmed in FIG. 1, application of colchicine of the present invention increased an expression amount of filaggrin concentration-dependently. The results show that a skin barrier is protected and also a prevention, improvement, and treatment effect for allergic skin diseases including atopic dermatitis is shown by the colchicine moisturizing effect according to the present invention.

(2) Confirmation of Moisture Loss, Skin Barrier Index, and Composite Index

Colchicine was continuously applied on back and ear skin areas of laboratory mice at concentration of 0.05% (w/w) twice a week for 4 weeks or more, and then a moisture loss and skin barrier and composite indexes were measured by a transdermal moisture loss meter (Tewameter).

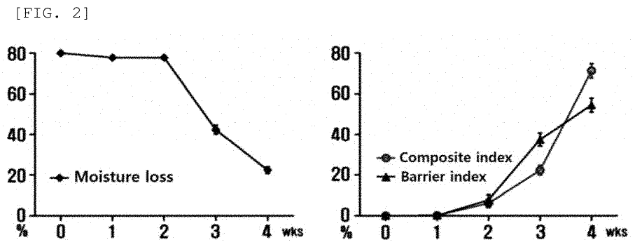

The experiment results are shown in FIG. 2, and were compared with an experiment animal group with no colchicine applied.

As confirmed in FIG. 2, it was found that since colchicine was applied on skin, a skin moisture loss was decreased to strongly show a moisturizing effect. In addition, since colchicine was applied, the skin composite index and barrier index were greatly increased.

From the above results, it was confirmed that the colchicine according to the present invention had an excellent moisturizing effect and a skin improvement effect. In addition, it was confirmed that an excellent prevention, improvement, and treatment effect for allergic skin diseases (in particular, atopic dermatitis) and/or pruritus cutaneous was shown. In addition, an additional advantage was shown in that the colchicine according to the present invention continuously improved a skin condition without any side effect even with long-term application for 4 weeks or more.

### Example 2. Confirmation of Allergic Skin Disease Improvement Effect of Colchicine

In order to prove a prevention, improvement, and treatment effect for allergic skin diseases of the compound according to the present invention, an allergic skin disease model (specifically, atopic dermatitis model) mouse derived by oxazolone was made.

Specifically, 150 μl of 3% 4-ethoxymethylene-2-phenyl-2-oxazolin-5-one (oxazolone) was applied on an abdominal skin of a 6-week-old laboratory animal to induce an immune response, and 20 μl of the same compound diluted to 0.1% was applied on the ear of a laboratory animal 3 times a week (total 6 times) to induce a skin disease. The ear of the laboratory animal was treated with 20 μl of the colchicine according to the present invention at a concentration of 0.05% 3 times a week (total 6 times). A negative control group treated with only ethanol which was a solvent and a positive control group treated with only oxazolone were also reflected in the experiment. The number of mice in each experiment group was maintained at 10 or more and the experiment was repeated 3 times.

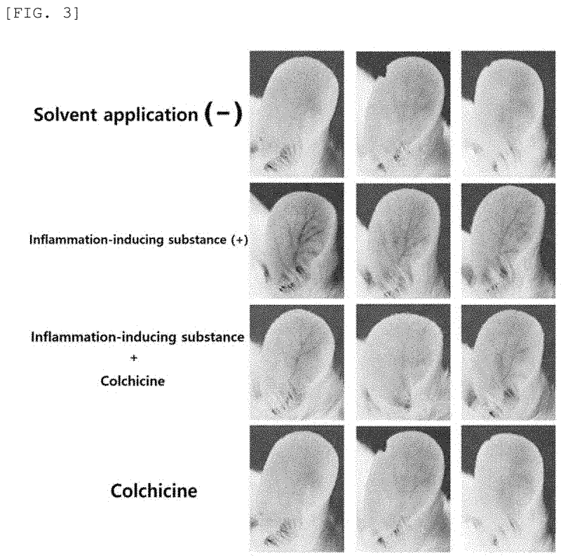

A change in skin which may be observed with the naked eye was shown specifically in FIG. 3.

As confirmed in FIG. 3, a red flare was shown on the ear of the mouse on which oxazolone was applied alone, and thus, it was found that an inflammatory response and an allergic reaction occurred.

However, a skin condition which was very similar to that of a normal group treated with only a solvent was observed in the animal group simultaneously treated with colchicine. This shows an excellent treatment effect of colchicine for allergic skin diseases.

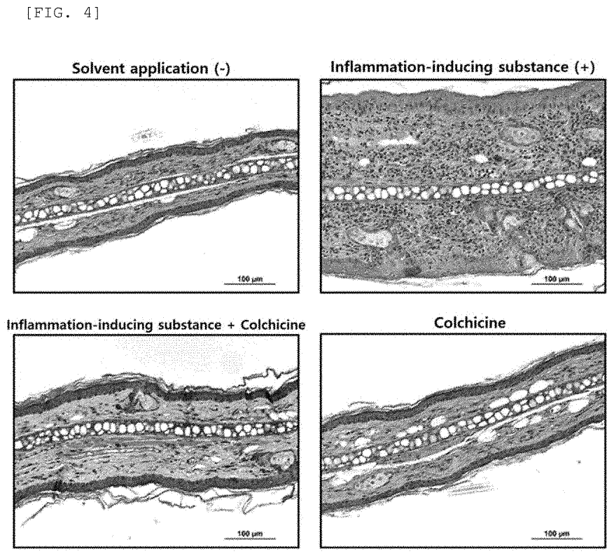

In addition, for pathological observation of skin after completing the application, an ear skin tissue of each mouse was collected and stained with Hematoxylin and Eosin, and the results are shown in FIG. 4.

As confirmed in FIG. 4, a pathological phenomenon of epidermal hyperplasia in which both a total skin thickness and an epithelial thickness were increased with treatment with oxazolone was observed. In particular, the total thickness and the epithelial thickness of the group treated with oxazolone were increased 3 times or more as compared with the skin of the group to which only ethanol was applied.

However, it was observed in the group to which colchicine was applied after the oxazolone treatment that the total skin thickness and the epithelial thickness were decreased to a degree very similar to the normal case. In addition, in the control group treated only colchicine also, a skin abnormal symptom was not detected.

From the above results, it was found that the colchicine application according to the present invention adjusted an inflammatory response in allergic skin diseases, thereby showing an excellent treatment effect.

### Example 3. Confirmation of Inflammatory Cytokine Inhibition Efficacy of Colchicine

After completing the application experiment of Example 2, a skin tissue was collected from each mouse and crushed with a tissue crusher to extract RNA. The crushed tissue powder was dissolved in a Trizol reagent solution (Sigma) and RNA was extracted using a RNeasy mini kit (Qiagen). The extracted RNA was reverse-transcribed to cDNA using ImProm-II™ Reverse Transcription kit (Promega), and then a cytokine degree present in the skin tissue was measured by a quantitative PCR (qPCR) method using CFX96™ Real-Time PCR Detection System (Bio-Rad).

Inflammatory cytokine which is most associated with the onset of allergic skin diseases such as atopic dermatitis is known as interleukin (IL)-4, IL-13, and the like which are mainly secreted in Th2 cells, and the expression change of the cytokine was confirmed.

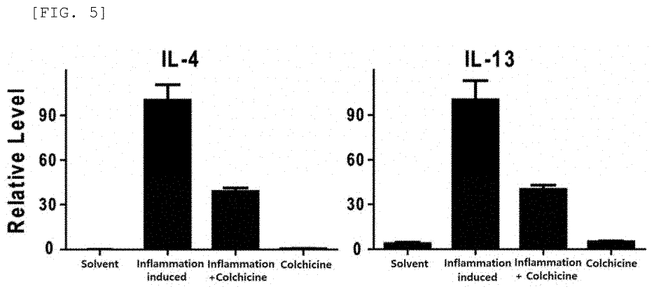

The results are shown in FIG. 5.

As confirmed in FIG. 5, the oxazolone treatment greatly increased the amounts of interleukin-4 (IL-4) and interleukin-13 (IL-13) which are inflammatory cytokines in tissues. However, the expression level of the cytokine in the group treated with both colchicine and oxazolone was significantly decreased to a degree of 50% or less. This result shows an excellent anti-inflammatory effect of colchicine.

A representative inflammatory cytokine target in a targeted research for allergic skin diseases which is mainly researched by global pharmaceutical companies is IL-4 and IL-13. The colchicine of the present invention showed an excellent effect in that IL-4 and IL-13 which are representative inflammatory cytokine commonly known to be highly associated with the onset of allergic skin diseases were greatly decreased.

The present invention has been described in detail in specific parts, and it is apparent that the detailed description is only a preferred embodiment to a person skilled in the art, without limiting the scope of the present invention thereto. Thus, the substantial scope of the present invention will be defined by the appended claims and their equivalents.

1. A method of treating an allergic skin disease in a subject in need thereof, pruritus cutaneous, or both of them, comprising administering a therapeutically effective amount of colchicine or a pharmaceutically acceptable salt thereof to the subject.

2. The method of claim 1, wherein the allergic skin disease is any one selected from the group consisting of atopic dermatitis, allergic dermatitis, and contact dermatitis.

3. The method of claim 2, wherein the allergic skin disease is atopic dermatitis.

4. The method of claim 1, wherein the administering is oral or parenteral administration.

5. The method of claim 4, wherein the parenteral administration is any one selected from the group consisting of transdermal administration, external application to skin, application to skin, intraperitoneal injection, intrarectal injection, subcutaneous injection, intravenous injection, intramuscular injection and intrathoracic injection.

6. The method of claim 5, wherein the parenteral administration is application to skin.

7. A method of claim 1, wherein the colchicine or the pharmaceutically acceptable salt thereof enhances expression of fillaggrin.

8. The method of claim 1, wherein the colchicine or the pharmaceutically acceptable salt thereof inhibits expression of IL-4 and IL-13.

9. The method of claim 1, wherein the colchicine or the pharmaceutically acceptable salt thereof decreases skin itching.

10. The method of claim 1, wherein the colchicine or a pharmaceutically acceptable salt thereof improves a skin barrier function.

11. The method of claim 6, wherein the colchicine or the pharmaceutically acceptable salt thereof has a formulation selected from the group consist of an ointment, a cream, a lotion, a gel, an external solution, a paste agent, a liniment and an aerosol agent.

12. A method of moisturizing skin of a subject, comprising applying colchicine or a cosmetically acceptable salt thereof to a skin of the subject.

13. The method of claim 12, wherein the colchicine or the pharmaceutically acceptable salt thereof enhances expression of fillaggrin.

14. The method of claim 12, wherein the colchicine or a pharmaceutically acceptable salt thereof improves a skin barrier function.

15. The method of claim 12, wherein the colchicine or the pharmaceutically acceptable salt thereof has a formulation selected from the group consisting of a solution, an external ointment, a cream, a foam, a nourishing lotion, a softening lotion, a pack, a softener, a milky lotion, a makeup base, an essence, a soap, a liquid detergent, a bath preparation, a sunscreen cream, a sun oil, a suspension, an emulsion, a paste, a gel, a lotion, a powder, a soap, a surfactant-containing cleansing, an oil, a powder foundation, an emulsion foundations, a wax foundations, a patch, and a spray.

16. The method of claim 15, wherein the formulation comprises 0.01 to 20 wt % of colchicine with respect to the total weight of the formulation.

17. (canceled)
